(19) United States
(12) Patent Application Publication
(10) Pub. No.: US 2013/0072580 A1
(43) Pub. Date: Mar. 21, 2013

(54) USE OF URINARY NGAL TO DIAGNOSE SEPSIS IN VERY LOW BIRTH WEIGHT INFANTS

(71) Applicants: Jonathan BARASCH, New York, NY (US); Elvira PARRAVICINI, Bronxville, NY (US); David A. BATEMAN, New Rochelle, NY (US); John M. LORENZ, New York, NY (US)

(72) Inventors: Jonathan BARASCH, New York, NY (US); Elvira PARRAVICINI, Bronxville, NY (US); David A. BATEMAN, New Rochelle, NY (US); John M. LORENZ, New York, NY (US)

(21) Appl. No.: 13/460,324

(22) Filed: Apr. 30, 2012

Related U.S. Application Data
(63) Continuation-in-part of application No. PCT/US2010/054811, filed on Oct. 29, 2010.
(60) Provisional application No. 61/256,288, filed on Oct. 29, 2009.

(51) Int. Cl. G01N 33/68

(52) U.S. Cl. CPC G01N 33/6893
USPC 514/789; 435/7.1; 435/287.2

(57) ABSTRACT

Methods for diagnosis of sepsis very low birth weight (VLBW) infants are disclosed. The diagnostic methods for sepsis are based on determining whether a bodily fluid sample, such as a urine sample, contains an amount of neutrophil gelatinase-associated lipocalin (NGAL) protein that exceeds or is less than a certain threshold level, or that falls within a certain range. The present invention also provides methods for monitoring the progression of sepsis, methods for monitoring sepsis treatment, methods of distinguishing between true-positive and false culture-positive sepsis, and kits for diagnosing sepsis in VLBW infants.

This application is a continuation-in-part of International Application No. PCT/US2010/054811 filed on Oct. 29, 2010, which claims the benefit of priority of U.S. patent application Ser. No. 61/256,288, filed Oct. 29, 2009, the contents of each of which are hereby incorporated by reference in their entireties.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### GOVERNMENT INTERESTS

This invention was made with government support under grant numbers DK-55388, DK-58872, and UL1 RR024156 awarded by the National Institute of Diabetes and Digestive and Kidney Diseases (NIDDK), the National Institutes of Health (NIH), and the United States Department of Health and Human Services (HHS). Accordingly, the United States Government has certain rights to the invention.

### BACKGROUND

Very low birth weight (VLBW) infants are at substantial risk of sepsis. Stoll et al., Pediatrics; 110: 285-91 (2002); Bizzarro et al., Pediatrics, 116(3): 595-602 (2005); Graham et al., Pediatr Infect Dis J, 25(2): 113-7 (2006); Fanaroff et al., Am J Obstet Gynecol, 196(2): 147.e1-8 (2007). The incidence of sepsis has recently been reported to be as high as 42% with a mortality rate of 18% in some neonatal intensive care unit (NICU) subpopulations. Brodie et al., Pediatr Infect Dis J, 19: 56-65 (2000).

The diagnosis of sepsis in premature infants is notoriously difficult due to the non-specific signs and symptoms in this population, the difficulties obtaining adequate samples for blood culture, and frequent co-morbid conditions that can mask, mimic, or accompany sepsis.

Neonatal sepsis is often classified as either early onset sepsis (onset typically occurring within 72 hours of birth) or late onset sepsis (onset typically occurring later than 72 hours after birth, e.g. 4-90 days after birth). Early onset sepsis is associated with acquisition of microorganisms from the mother whereas late onset sepsis is associated with acquisition of microorganisms after birth. Different microorganisms may be associated with early and late onset sepsis. The microorganisms most commonly associated with early-onset infection include group B Streptococcus (GBS), Escherichia coli, coagulase-negative Staphylococcus, Haemophilus influenzae, and Listeria monocytogenes. Organisms that have been implicated in causing late-onset sepsis syndrome include coagulase-negative staphylococci, Staphylococcus aureus, E coli, Klebsiella, Pseudomonas, Enterobacter, Candida, GBS, Serratia, Acinetobacter, and anaerobes.

Neutrophil gelatinase-associated lipocalin (NGAL) is a 25-kDa protein expressed at very low level in neutrophils and several human tissues, including the lung, gastrointestinal tract, and kidney. Expression of this protein rises dramatically when epithelial organs undergo signaling, which is usually associated with cell damage, including ischemia-reperfusion injury, Mishra et al., J Am Soc Nephrol, 14(10): 2534-43 (2003); cytotoxins; Mishra et al., J Am Soc Nephrol, 14(10): 2534-43 (2003); and sepsis, Schmidt-Ott et al., Curr Opin Nephrol Hypertens, 15: 442-449 (2006). In these circumstances, NGAL is upregulated in the circulation and in the urine. The source of urinary NGAL (uNGAL) is predominately the kidney tubule. Schmidt-Ott et al., Curr Opin Nephrol Hypertens, 15: 442-449 (2006); Schmidt-Ott et al., J Am Soc Nephrol, 18: 407-413 (2007).

A need exists for developing a diagnosis of sepsis in infants, such as very low birth weight infants. Such a diagnosis can be critical in preventing morbidity and mortality in this fragile population.

### SUMMARY OF THE INVENTION

The present invention is based, in part, on certain discoveries which are described more fully in the Examples section of the present application. For example, the present invention is based, in part, on the discovery that levels of NGAL protein in the urine in VLBW infants with culture-positive sepsis are much higher than the levels of NGAL in the urine of control patients. The present invention also provides diagnostic kits for diagnosing sepsis in VLBW infants.

In one embodiment, the present invention provides a method for determining whether a very low birth weight (VLBW) infant has sepsis, the method comprising determining the concentration of NGAL protein in a urine sample from a very low birth weight infant, wherein a concentration of NGAL in the urine sample that exceeds a threshold amount indicates that the infant has sepsis, and wherein a concentration of NGAL in the urine sample that is less than the threshold amount indicates that the infant does not have sepsis. In some such embodiments, the method comprises subsequently treating the VLBW infant with antibiotics.

In another embodiment, the present invention provides a method for determining whether a VLBW infant has late onset sepsis, the method comprising determining the concentration of NGAL protein in a urine sample from a very low birth weight infant, wherein a concentration of NGAL in the urine sample that exceeds a threshold amount indicates that the infant has late onset sepsis, and wherein a concentration of NGAL in the urine sample that is less than the threshold amount indicates that the infant does not have late onset sepsis. In some such embodiments, the method comprises subsequently treating the VLBW infant with antibiotics, such as those suited to treatment of late onset sepsis in particular.

In another embodiment, the present invention provides a method for distinguishing between true sepsis (e.g. late onset sepsis) and false-positive sepsis (e.g. late onset sepsis) in a VLBW infant, the method comprising determining the concentration of NGAL protein in a urine sample from a VLBW infant with sepsis, wherein a concentration of NGAL in the urine sample that exceeds a threshold amount indicates that the infant has true sepsis (e.g. late onset sepsis), and wherein a concentration of NGAL in the urine sample that is less than the threshold amount indicates that the infant has false-positive sepsis (e.g. late onset sepsis). In some such embodiments, the method comprises subsequently treating the VLBW infant with antibiotics, such as those suited to treatment of late onset sepsis in particular.

In another embodiment, the present invention provides a method for monitoring the progression of sepsis (e.g. late onset sepsis) in a VLBW infant, the method comprising: obtaining a first urine sample from a VLBW infant at a first time point; obtaining a second urine sample from the VLBW infant at a second time point that is after the first time point; and determining the amount of NGAL protein in the first and second urine samples, wherein an amount of NGAL protein in the first urine sample that exceeds the amount in the second urine sample indicates that the sepsis (e.g. late onset sepsis) is improving, and wherein an amount of NGAL protein in the second urine sample that exceeds the amount in the first urine sample indicates that the sepsis (e.g. late onset sepsis) is worsening. In some such embodiments, the method further comprises subsequently treating the VLBW infant with antibiotics. In some such embodiments the first urine sample is obtained before initiation antibiotic treatment, and the second urine sample is obtained after initiation of antibiotic treatment. In other embodiments both the and second urine samples are obtained after initiation of antibiotic treatment. The method may further comprising subsequently adjusting the infant's treatment regimen based on whether the sepsis is improving or worsening under the existing treatment regimen.

The present invention also provides diagnostic kits for determining whether a VLBW infant has sepsis, the kits comprising: a device for detecting NGAL protein in the urine; a positive control containing NGAL protein; and instructions indicating threshold level of NGAL above which a diagnosis of sepsis can be made, or above which a diagnosis of late onset sepsis in particular can be made, in a VLBW infant. In some embodiments, the diagnostic kits comprise an antibody that binds to the NGAL protein.

These and other embodiments of the invention are further described in the following sections of the application, including the Detailed Description, Examples, Claims, and Drawings.

### BRIEF DESCRIPTION OF THE FIGURES

FIG. 1 shows the geometric mean uNGAL concentrations and 95% confidence intervals on the day that blood culture was drawn in VLBW infants with late onset culture positive sepsis (A), a single positive blood culture for Staphylococcus epidermidis (S. epidermidis) (B), a negative blood culture with ≧7 days of antibiotic treatment (C), and a negative blood culture with <7 days of antibiotic treatment (D). The dashed line represents the upper bound of the 95% confidence interval of uNGAL from VLBW infants with uncomplicated clinical courses. Huynh et al., Pediatr Res (Aug. 12, 2009).

FIG. 2 shows daily geometric mean uNGAL concentrations and 95% confidence intervals for VLBW infants with culture-positive sepsis from 5 days prior to 5 days after the positive blood culture. The dashed line represents the upper bound of the 95% confidence interval of uNGAL from VLBW infants with uncomplicated clinical courses. Huynh et al., Pediatr Res (Aug. 12, 2009). Significant differences of each day from day −1 are denoted as * for P<0.05, ** for P<0.005, *** for P<0.0005.

FIG. 3 shows a receiver operator curve (ROC) analysis for uNGAL on the day blood cultures are drawn, and shows the ability of uNGAL to discriminate between episodes of culture proven sepsis (group A) and episodes of negative blood culture treatment <7 d (Group D) for both males and females. AUC and 95% CI for both sexes combined are 0.87 and 0.77-0.97, respectively.

FIG. 4 shows a bar graph of geometric mean uNGAL concentrations for different culture result categories.

FIG. 5 shows a receiver operator curve (ROC) analysis for culture positive versus culture negative evaluation.

FIG. 6 provides a bar graph showing the number of patients per culture result category.

### DETAILED DESCRIPTION

The present invention is based, in part, on certain discoveries which are described more fully in the Examples section of the present application. For example, the present invention is based, in part, on the discovery that levels of NGAL protein in the urine in VLBW infants with culture-positive sepsis are much higher than the levels of NGAL in the urine of control patients. The present invention also provides diagnostic kits for diagnosing sepsis in VLBW infants. Because the diagnosis of sepsis in infants is nearly impossible to confirm at the time when clinical suspicion first arises, the present invention is useful for an early diagnosis of sepsis.

### Abbreviations and Definitions

The abbreviation “NGAL” refers to Neutrophil Gelatinase Associated Lipocalin. NGAL is also referred to in the art as human neutrophil lipocalin, siderocalin, a-micropglobulin related protein, Scn-NGAL, lipocalin 2, 24p3, superinducible protein 24 (SIP24), uterocalin, and neu-related lipocalin. These alternative names for NGAL may be used interchangeably herein. Unless stated otherwise, the term “NGAL,” as used herein, includes any NGAL protein, fragment, or mutant that is expressed in the kidney, and which can be detected in a bodily fluid such as urine. In some embodiments the NGAL protein is wild-type human NGAL.

The abbreviation “uNGAL” is urinary NGAL and refers to NGAL in the urine.

The abbreviation “VLBW” is very low birth weight. The term VLBW infant is an art used term and is used herein in accordance with its normal meaning in clinical medicine. For example, the term VLBW infant is generally used to refer to an infant whose weight at birth is less than 3 pounds, 5 ounces or 1,500 grams).

The term “sepsis” is used herein in accordance with its normal meaning in clinical medicine, and includes, for example systemic and or blood borne infections, such as bacterial infections. Neonatal sepsis is often classified as either early onset sepsis (onset typically occurring within 72 hours of birth) or late onset sepsis (onset typically occurring later than 72 hours after birth, e.g. 4-90 days after birth).

The abbreviation “ROC” refers to receiver operating characteristic. ROC curves are widely used in the art for assessing diagnostic and prognostic tests, for example using different threshold or cut-off values. See, for example, Zweig & Campbell, (1993), “Receiver-operating characteristic (ROC) plots: a fundamental evaluation tool in clinical medicine”. Clinical chemistry 39 (8): 561-577; and Zou et al., (2007). “Receiver-operating characteristic analysis for evaluating diagnostic tests and predictive models.” Circulation, 6; 115(5): 654-7; and Lasko et al., (2005), “The use of receiver operating characteristic curves in biomedical informatics.” Journal of Biomedical Informatics, 38(5):404-415, the contents of each which are hereby incorporated by reference.

The abbreviation “AUC” refers to area under the curve, e.g. area under the receiver operating characteristic (ROC) curve.

The term “about” is used herein to mean approximately, roughly, around, or in the region of When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### Description

In one aspect of the invention, levels of NGAL protein in a bodily fluid, such as urine, that exceed a certain threshold amount can be used to diagnose sepsis. It is a discovery of the invention that, in the study performed (see Example 1), mean uNGAL in VLBW infants with culture-positive sepsis (group A) (179 ng/ml, 95% CI [100 ng/ml, 318 ng/ml]) was significantly higher compared with the previously determined reference range for VLBW infants (mean 6.5 ng/ml, upper bound of 95% CI 55 ng/ml) and compared to the means in the other three test groups without culture-positive sepsis (single culture positive for S. epidermis; negative culture with antibiotic treatment for more than or equal to seven days; and negative culture with antibiotic treatment for less than seven days). Infants with culture-positive sepsis expressed uNGAL at levels about 30 fold higher than healthy infants.

Accordingly, in one embodiment, the present invention provides methods for determining whether a VLBW infant has sepsis (e.g. late onset sepsis), the method comprising determining the concentration of NGAL protein in a urine sample from a VLBW infant, wherein a concentration of NGAL in the urine sample that exceeds a threshold amount indicates that the infant has sepsis (e.g. late onset sepsis), and wherein a concentration of NGAL in the urine sample that is less than the threshold amount indicates that the infant does not have sepsis (e.g. late onset sepsis).

In one embodiment, the threshold amount for determining whether a VLBW infant has sepsis is between about 10 ng/ml and about 200 ng/ml, or between about 25 ng/ml and about 150 ng/ml. In one embodiment, the threshold amount for determining whether a VLBW infant has sepsis is between about or between about 60 ng/ml and 150 ng/ml, or between about 65 ng/ml and 150 ng/ml, or between about 70 ng/ml and 150 ng/ml, or between about 75 ng/ml and 150 ng/ml, or between about 80 ng/ml and 150 ng/ml, or between about 85 ng/ml and 150 ng/ml, or between about 90 ng/ml and 150 ng/ml, or between about 95 ng/ml and 150 ng/ml, or between about 100 ng/ml and 150 ng/ml, or between about 105 ng/ml and 150 ng/ml, or between about 110 ng/ml and 150 ng/ml, or between about 115 ng/ml and 150 ng/ml, or between about 120 ng/ml and 150 ng/ml, or between about 125 ng/ml and 150 ng/ml, or between about 130 ng/ml and 150 ng/ml. In each of the previous ranges, the upper limit of the range can be about 150 ng/ml, or about 145 ng/ml, or about 140 ng/ml, or about 135 ng/ml, or about 130 ng/ml, or about 125 ng/ml, or about 120 ng/ml, or about 115 ng/ml, or about 110 ng/ml, or about 105 ng/ml, or about 100 ng/ml.

In another embodiment, the threshold amount for determining whether a VLBW infant has sepsis is about 50 ng/ml, or about 60 ng/ml, about 70 ng/ml, or about 80 ng/ml, or about 90 ng/ml, or about 100 ng/ml, or about 110 ng/ml, or about 120 ng/ml, or about 130 ng/ml, or about 140 ng/ml, or about 15 ng/ml. In one embodiment, the threshold amount is about 75 ng/mL in VLBW males and about 130 ng/mL in VLBW females. Unless specified, the threshold values indicated herein are applicable to both males and females.

In another embodiment, the threshold amount for determining whether a VLBW infant has sepsis is about 10 ng/ml, or about 20 ng/ml, about 25 ng/ml, or about 30 ng/ml, or about 35 ng/ml, or about 40 ng/ml, or about 45 ng/ml, or about 50 ng/ml, or about 55 ng/ml, or about 60 ng/ml, or about 65 ng/ml, or about 70 ng/ml, or about 75 ng/ml, or about 80 ng/ml or about 85 ng/ml or about 90 ng/ml or about 95 ng/ml or about 100 ng/ml, or about 110 ng/ml, or about 120 ng/ml, or about 130 ng/ml, or about 140 ng/ml, or about 150 ng/ml, or about 160 ng/ml, or about 170 ng/ml, or about 180 ng/ml, or about 190 ng/ml, or about 200 ng/ml.

In other embodiments the threshold may be selected by analysis of statistical data, e.g. using ROC and AUC analysis, in order to select a threshold having the desired specificity, sensitivity, positive predictive value, and negative predictive value.

In another embodiment, the present invention provides methods for determining whether a VLBW infant has late onset sepsis or early onset sepsis, the method comprising determining the concentration of NGAL protein in a urine sample from a VLBW infant, wherein a concentration of NGAL in the urine sample that exceeds a threshold amount indicates that the infant has late onset sepsis, and wherein a concentration of NGAL in the urine sample that is less than the threshold amount indicates that the infant has early onset sepsis. The thresholds may be any of those set forth above.

In another embodiment, the present invention provides methods for determining whether a VLBW infant has true positive sepsis (e.g. late onset sepsis) or false positive sepsis. This embodiment is based, in part, on the discovery described in the Examples that uNGAL levels are significantly higher in VLBW infants in whom one or more blood cultures is positive for a pathogen or two or more blood cultures are positive for Staphylococcus epidermidis (S. epidermidis), as compare to VLBW infants having only a single positive culture for S. epidermidis. S. epidermidis is a coagulase negative bacterium present on the skin that can contaminated blood samples as a result of needles contacting S. epidermidis on the skin. Thus, it is frequently the case that VLBW infants having only a single positive culture for S. epidermidis do not have sepsis but that instead the blood culture was contaminated, for example during the process of taking the blood sample, thus giving a “false-positive” result. The present invention provides methods for determining whether a VLBW infant has true positive sepsis (e.g. late onset sepsis) or false-positive sepsis (e.g. late onset sepsis), the method comprising determining the concentration of NGAL protein in a urine sample from a VLBW infant, wherein a concentration of NGAL in the urine sample that exceeds a threshold amount indicates that the infant has true positive sepsis, and wherein a concentration of NGAL in the urine sample that is less than the threshold amount indicates that the infant has early onset sepsis. The thresholds may be any of those set forth above.

In another embodiment, the present invention provides methods for determining whether a sepsis treatment regimen in a VLBW infant is effective, the method comprising taking at least a first urine sample and a second urine sample from the infant, the first urine sample being taken at a period earlier in time than the later urine sample, wherein a concentration of NGAL in the second urine sample that is less than the concentration of NGAL in the first urine sample indicates that the treatment is effective, and wherein a concentration of NGAL in the second urine sample that is greater than the concentration of NGAL in the first urine sample indicates that the treatment is not effective. The thresholds may be any of those set forth above.

In another embodiment, the present invention provides methods for determining whether and infant's sepsis is worsening or improving, the method comprising taking at least a first urine sample and a second urine sample from the infant, the first urine sample being taken at a period earlier in time than the later urine sample, wherein a concentration of NGAL in the second urine sample that is less than the concentration of NGAL in the first urine sample indicates that the infant's sepsis is improving, and wherein a concentration of NGAL in the second urine sample that is more than the concentration of NGAL in the second urine sample indicates that the infant's sepsis is worsening. The thresholds may be any of those set forth above.

In some embodiments, the step of determining the amount of NGAL in the urine can comprise performing an immunoassay to detect NGAL protein.

In some embodiments, the methods further comprise selecting or adjusting the infant's treatment regimen based on whether the concentration of NGAL in the urine sample exceeds or is less than the threshold amount, or whether the amount of NGAL is increasing or decreasing over time. In some embodiments, the methods further comprise selecting or adjusting the infant's treatment regimen based on whether the concentration of NGAL in the urine sample indicates that the infant has early or late onset sepsis. In some embodiments, the step of selecting or adjusting the infant's treatment regimen can comprise selecting or adjusting the antibiotic or combination of antibiotics to be used in treating the sepsis and/or selecting or adjusting the dosage, duration, or other parameter of such treatment.

In some embodiments a urine sample to be tested for NGAL is taken at the time that sepsis is first suspected. In some embodiments a urine sample to be tested is taken within 1 day of birth. In some embodiments the urine sample to be tested is taken within 2 days of birth. In some embodiments a urine sample to be tested is taken within 3 days of birth. In some embodiments a urine sample to be tested is taken within 4 days of birth. In some embodiments a urine sample to be tested is taken within 5 days of birth. In some embodiments a urine sample to be tested is taken within 6 days of birth. In some embodiments a urine sample to be tested is taken within 7 days of birth. In some embodiments a urine sample to be tested is taken within 8 days of birth. In some embodiments a urine sample to be tested is taken within 9 days of birth. In some embodiments a urine sample to be tested is taken within 10 days of birth. In some embodiments a urine sample to be tested is taken within 11 days of birth. In some embodiments a urine sample to be tested is taken within 12 days of birth. In some embodiments a urine sample to be tested is taken within 13 days of birth. In some embodiments a urine sample to be tested is taken within 14 days of birth. In some embodiments a urine sample to be tested is taken within three weeks of birth. In some embodiments a urine sample to be tested is taken within four weeks of birth. In some embodiments a urine sample to be tested is taken within four weeks of birth. In some embodiments a urine sample to be tested is taken within four weeks of birth. In some embodiments a urine sample to be tested is taken within two months of birth. In some embodiments a urine sample to be tested is taken within three months of birth.

In embodiments of the invention wherein multiple urine samples are taken, the interval between the samples may be hours, days, weeks, or months. For example, in some embodiments, urine sample are taken every 12 hours, or daily. One of skill in the art can select a suitable interval between the urine samples depending on factors such as the infant's age, severity of infection, the treatment regimen, etc.

In other embodiments, the methods described herein can be used in conjunction with other methods used for the diagnosis of sepsis, including culture-based detection methods (blood or urine cultures), or including tests based on quantitative C-reactive protein (CRP) measurement. In such situations, the NGAL measurements can provide a means for distinguishing true positives from false positives, and a means for distinguishing true negatives from false negatives. For example, in one embodiment if an infant has a positive result in culture-based test for sepsis but the level of NGAL in the urine sample is less than one of the threshold values set forth herein, then the positive culture result can be a false positive result. Similarly, in another embodiment if an infant has a negative result in culture-based test for sepsis but the level of NGAL in the urine sample exceeds one of the threshold values set forth herein, then the negative culture result can be a false negative result. Conversely, in another embodiment if an infant has a negative result in culture-based test for sepsis and the level of NGAL in the urine sample is less than one of the threshold values set forth herein, then the negative culture result can be a true negative result. Similarly, in another embodiment if an infant has a positive result in culture-based test for sepsis and the level of NGAL in the urine sample exceeds one of the threshold values set forth herein, then the positive culture result can be a true positive result.

In another embodiment, the present invention provides diagnostic kits for use in any of the above methods described herein the kit comprising: (a) a device for detecting NGAL protein in the urine; (b) a positive control containing NGAL protein; and (c) instructions indicating a threshold level of NGAL above which a diagnosis of sepsis can be made.

In one embodiment, the device in the diagnostic kits comprises an antibody that binds to the NGAL protein. In another embodiment, the device in the diagnostic kits comprises an ELISA plate, a urine dipstick, or a test strip.

As described above, in certain embodiments, the present invention provides various methods forbased on determining whether the amount of NGAL in a urine sample exceeds or is less than a certain threshold amount. In addition to the threshold levels specified herein, a threshold level can also be selected by reviewing the data provided in the Examples section of this application, so that the threshold level is sufficiently high that it is more likely than not that a VLBW infant having that level of uNGAL will have sepsis. In all of the embodiments above that deal with making an assessment relating to sepsis based on detecting a level of NGAL in the urine that exceeds a threshold amount, ranges of uNGAL amounts can be used in the place of threshold values. Also, the upper end of each of the preceding ranges can be adjusted. Thus, threshold levels or ranges of NGAL other than those specifically described herein may be used in accordance with the invention.

It is a discovery of the invention that NGAL levels are higher in the urine of VLBW infants with sepsis (including late onset sepsis) as compared to infants without sepsis. The mean levels of uNGAL in such groups (VLBW controls, VLBW infants with sepsis) may vary in different groups of infants or depending on the methodology used to measure NGAL levels. Accordingly, the present invention provides for the general concept of using uNGAL levels to diagnose sepsis (e.g. late onset sepsis) in VLBW infants, and not only methods that rely on the specific thresholds and ranges provided herein.

In certain embodiments, the NGAL protein detected and/or measured in the methods of the present invention has an amino acid sequence as defined by one of the following GenBank accession numbers, NP_{—}005555 (human NGAL), CAA67574 (human NGAL), P80188 (human NGAL), AAB26529 (human NGAL), P11672 (mouse NGAL), P30152 (rat NGAL), AAI132070 (mouse NGAL), AAI132072 (mouse NGAL), AAH33089 (human NGAL), and CAA58127 (human NGAL), or is a homolog, variant, derivative, fragment, or mutant thereof, and/or has at least 80% sequence identity, e.g., 85%, 90%, 95%, 98% or 99% sequence identity, with one of the above sequences.

In certain embodiments of the invention, it can be desirable to use a positive control for detecting NGAL. NGAL protein for use as a positive control can be obtained from any source or produced by any method known in the art. For example, NGAL protein can be recombinantly produced. Methods for the recombinant production of proteins are well known in the art. For example, a nucleotide sequence encoding NGAL can be included in an expression vector containing expression control sequences and expressed in, and purified from, any suitable cell type, such as bacterial cells or mammalian cells. For example, for use as a positive control in the methods of the invention, recombinant NGAL can be produced as described in Yang, et al. (2002) Mol Cell 10, 1045-1056; Goetz et al. (2002) Mol. Cell 10, 1033-1043; Goetz et al. (2000) Biochemistry 39, 1935-1941; and Mori, et al. (2005) J. Clin Invest. 115, 610-621, the contents of which are hereby incorporated by reference.

Although the amounts of NGAL described herein are generally referred to in terms of the amount of NGAL by concentration (e.g. in ng/mL), NGAL can also be measured and/or represented in other units, including but not limited to measurements of the amount of NGAL by mass (e.g. in nanograms or micrograms), the amount by mass of NGAL relative to the amount by mass of urine creatinine (UCr), e.g. NGAL pg/g creatinine, or any other units. It should be understood that amounts of NGAL measured and/or represented in other units can be equivalent to the amounts and ranges described herein in terms of ng/mL. One of ordinary skill in the art can readily make the necessary conversions between units.

According to the methods of the invention, samples of a bodily fluid can be obtained and/or tested using any means. For example, methods for collecting, handling and processing bodily fluids such as urine are well known in the art and can be used in the practice of the present invention. In some embodiments, it is not necessary to obtain and keep a sample of the bodily fluid from the infant. For example, in some embodiments, the infant can urinate onto a test strip, for example, a test strip of the type used in pregnancy testing kits.

Although generally the sample of a bodily fluid such as urine is obtained from an infant and tested by a laboratory or by a medical professional (for example using an automated urinalysis machine configured to test for NGAL, or an nNGAL testing kit, e.g. a urine dipstick based kit, or an ELISA based kit), home-testing kits are also within the scope of the present invention. In one aspect, the present invention comprises a kit for performing the methods of the invention, containing, for example, a device for detecting NGAL protein in the urine, and optionally including a positive control containing NGAL protein, and optionally including instructions, for example regarding the threshold levels of NGAL above which a diagnosis of sepsis can be made. The device in such kits can comprise, for example, an ELISA plate, a dipstick or a test strip to be dipped in a urine sample or to have a sample or urine applied thereto, or a stick on which the subject should urinate. In some embodiments, such devices are configured such that they give a positive result only if the level of NGAL exceeds a threshold level, such as one of the threshold levels described herein. Methods for making and using such devices are well known in the art. Kits (ELISA kits), antibodies, and other reagents for detection of NGAL in the urine are commercially available, e.g. from Bioporto Diagnostics A/S and from R & D Systems, and can be used to make a kit according to the present invention. Such kits can be used by, for example, the infants' parents or can be used by medical or laboratory staff.

According to the methods of the invention, the presence and/or amount of NGAL protein in a bodily fluid, such as urine, can be detected and/or measured using any means known in the art. For example, in one embodiment, NGAL protein can be detected using antibodies that are specific to NGAL. Any antibody, such as a monoclonal or polyclonal antibody, that binds to NGAL can be used. For example, monoclonal antibodies that bind to NGAL are described in “Characterization of two ELISAs for NGAL, a newly described lipocalin in human neutrophils”, Lars Kjeldsen et al., (1996) Journal of Immunological Methods, Vol. 198, 155-16, the contents of which are herein incorporated by reference. An example of a polyclonal antibody for NGAL is described in “An Iron Delivery Pathway Mediated by a Lipocalin”, Jun Yang et al., Molecular Cell, (2002), Vol. 10, 1045-1056, herein incorporated by reference in its entirety.

Any method can be used to detect and/or measure the levels of NGAL protein, including, but not limited to, immunohistochemistry-based methods, immunoblotting-based methods, immunoprecipitation-based methods, affinity-column based methods (including immunoaffinity column based methods), ELISA-based methods, other methods in which an NGAL antibody is immobilized on a solid substrate (such as beads), and the like. In some such methods the antibody to NGAL, or a secondary or tertiary antibody that binds directly or indirectly to the NGAL antibody, can be labeled with a detectable moiety, such as a fluorescent moiety, a radioactive moiety, or a moiety that is an enzyme substrate and can be used to generate a detectable moiety, such as horse radish peroxidase. Such methods are well known in the art and can be used to detect the presence and/or measure the amount of NGAL in a bodily fluid sample, such as urine, without undue experimentation.

In circumstances where the amount of NGAL is to be measured, positive controls containing known amounts of NGAL protein can be used, for example, for calibration purposes. NGAL protein for use as a positive control can be obtained from any source or produced by any method known in the art. For example, NGAL protein can be recombinantly produced. Methods for the recombinant production of proteins are well known in the art. For example, a nucleotide sequence encoding NGAL can be included in an expression vector containing expression control sequences and expressed in, and purified from, any suitable cell type, such as bacterial cells or mammalian cells. For example, for use as a positive control in the methods of the invention, recombinant NGAL can be produced as described in Yang, et al. (2002) Mol Cell 10, 1045-1056; Goetz et al. (2002) Mol. Cell 10, 1033-1043; Goetz et al. (2000) Biochemistry 39, 1935-1941; and Mori, et al. (2005) J. Clin Invest. 115, 610-621, the contents of which are hereby incorporated by reference.

Those skilled in the art will recognize, or be able to ascertain, using no more than routine experimentation many equivalents to the specific embodiments of the invention described herein. Such equivalents are intended to be within the scope of the present invention.

The invention is further described by the following non-limiting Examples.

### EXAMPLES

### Example 1

Very low birth weight (VLBW) infants are at substantial risk of late onset (occurring after day of life four) sepsis. The incidence of sepsis has recently been reported to be as high as 42% with a mortality rate of 18% in some NICU subpopulations. The diagnosis of sepsis in premature infants is notoriously difficult because of its nonspecific signs and symptoms in this population, the difficulties obtaining adequate samples for blood culture, and frequent co-morbid conditions that can mask, mimic, or accompany sepsis. As a result, empiric antibiotic therapy for late onset sepsis is frequently prescribed with the potential for concomitant toxicity and the emergence of resistance.

Several early biomarkers for sepsis in the neonatal population have been proposed. Quantitative C-reactive protein (CRP) measurement is widely used in sepsis screening in newborn infants. CRP sensitivity is low at the time of first clinical suspicion but improves with two repeated measurements at 12-24 h intervals. However, its positive predictive value remains low, and it is less reliable in preterm infants. Many other cytokines or acute phase reactants have also been studied, but similarly lack sensitivity unless measured serially or in combination with other tests.

An ideal biomarker for infection in newborns should be quantifiable using a small, easily obtained body fluid sample; be elevated early in the course of infection; and have an adequate window of opportunity for sampling, i.e., a sufficient period during which its level remains abnormal. In addition, the marker should be biochemically stable and have minimal transportation requirements. The analytical procedure should have a short turnaround time, low cost, and be available around the clock. The results should be comparable among different laboratories. Most importantly, a clinically useful infection marker must have a well defined threshold value for differentiating infected from noninfected infants and be able to identify infected infants at an early stage. A very high sensitivity and negative predictive value (approaching 100%) and good specificity and positive predictive value (>85%) are desirable. The ability to differentiate various types of infection (bacterial, fungal, or viral) would be advantageous. Further, it should be able to reflect the progress of the disease and guide the use of antimicrobial therapy.

### Study Subjects

An observational study was conducted of VLBW infants (birth weight ≦1500 grams) Subjects were excluded if they were small for gestational age (SGA), had major congenital anomalies or abnormal karyotype, or the study team anticipated early death or transfer to a referring institution. NGAL Assay

Bagged spot urine specimens (≧0.5 ml) were collected on the day of enrollment and daily until 32 weeks post-menstrual age, discharge, or death—which ever occurred first. The assay employed has been previously described. Huynh et al. Pediatr Res (Aug. 12, 2009). Briefly, urine was centrifuged (5000 rpm, 5 minutes) and supernatant stored at −80° C. NGAL concentration was determined by immunoblot. Urinary NGAL analyses were performed in batches days to weeks after collection, and medical caregivers were unaware of the results.

### Case Definitions and Data Collection

Late onset culture positive sepsis was defined as presence of one or more blood cultures positive for a pathogen or two or more blood cultures positive for Staphylococcus epidermidis (S. epidermidis) after day of life four. Episodes of late onset culture positive sepsis were ascertained by daily review of the clinical course of each infant, together with review of their electronic medical records. In addition to episodes of culture proven sepsis (group A), three additional categories of suspected sepsis were identified for comparison: B) episodes of single positive blood cultures for S. epidermidis; C) episodes with negative blood cultures and ≧7 days of antibiotic therapy; and D) episodes with negative blood cultures with <7 days of antibiotic therapy. Episodes of sepsis-related acute kidney injury (AKI), defined as an increase in serum creatinine ≧0.3 mg/dl sustained for at least 48 hours during treatment for culture positive sepsis, were also identified.

Serum creatinine was measured by Jaffe kinetic reaction as per the standard of care. Sepsis evaluations were undertaken at the discretion of the attending medical team.

### Statistical Analysis

Mean uNGAL concentrations of group A on day 0 and day −1 were compared with the values of healthy VLBW infants previously reported (Huynh et al. Pediatr Res (Aug. 12, 2009)) and with the values of the other three groups of episodes of suspected sepsis (B, C and D) using linear models adjusted for birth weight and gender. Linear mixed models with random intercepts for subjects, where the outcome modeled was log(uNGAL), were used to estimate the mean and 95% confidence interval for log(uNGAL) on each day from 5 days before to 5 days after the day that blood cultures were obtained (day 0) in infants with culture proven sepsis (group A); results were exponentiated back to absolute scale. Therefore “mean uNGAL” values reported from regression models as well as throughout are geometric means. Results from these models were used to delineate the pattern of mean uNGAL over the time interval ±5 days from the day of blood cultures, using the value on day −1 as reference.

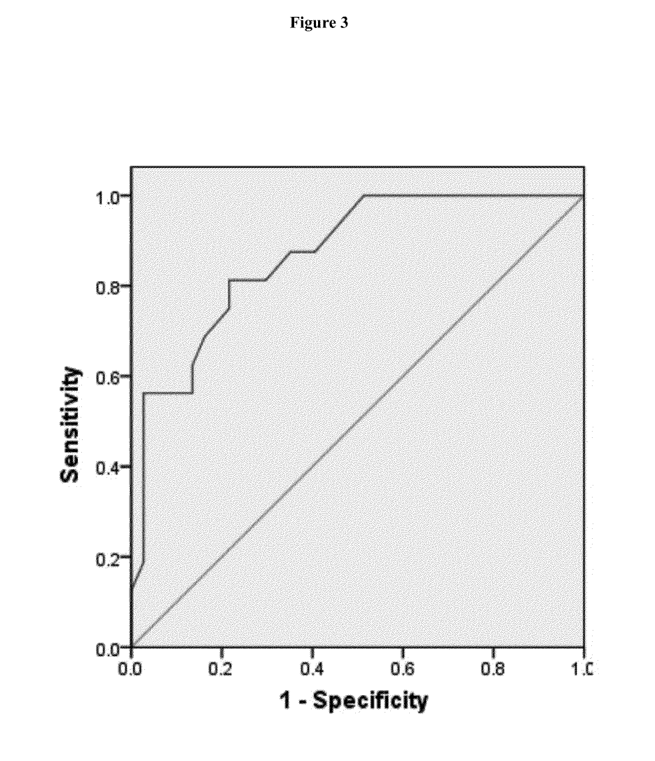

Receiver-operator curves (ROC) were constructed to evaluate the ability of uNGAL to discriminate between episodes of confirmed sepsis (group A) and episodes with negative blood culture and treatment <7 d (group D) on DOL 0, for sexes combined (shown in FIG. 3) and for males and females separately using SPSS software (SPSS Inc, Chicago Ill. 60606, www.spss.com). Areas under the curve (AUC) values and their 95% CIs were determined for sexes combined and separately. To estimate the sensitivity and specificity of a hypothetical screening test based on current data, uNGAL values at the 99 percentile of the reference ranges for males and females (20) were combined into a single indicator variable and cross-tabulated against the results of confirmed positive and confirmed negative blood cultures (groups A and D).

### Results

Ninety-one VLBW infants were recruited for this study within the first 7 days of life. he reference range for uNGAL concentrations in VLBW infants was determined from daily urine samples taken from 50 patients with uncomplicated clinical courses as previously reported. Huynh et al. Pediatr Res (Aug. 12, 2009). In nearly all cases, vancomycin and gentamicin were initially administered and then antibiotic therapy was adjusted as appropriate based on culture results.

Clinical characteristics of infants in the four sepsis groups are shown in Table 1. Sixteen infants (group A) had 16 episodes of culture-proven sepsis: 8 due to S. aureus, 2 due to S. epidermidis, 3 due to gram-negative bacilli, and 3 due to Candida species. Of these 16, three had associated AKI; none had necrotizing enterocolitis.

TABLE 1
Clinical characteristics of study infants according to suspected sepsis
group and infants with uncomplicated clinical course previously reported. (Huynh et al.
Pediatr Res (Aug. 12, 2009)).

| | | | B: Single | C: Culture | D: Culture |
|---|---|---|---|---|---|
| | Uncomplicated | A: Culture | Positive | Negative, | Negative, |
| | Clinical | Positive | Culture for | Treated | Treated |
| | Course | Sepsis | S. epidermidis | ≧7 days | <7 days |
| Number of | 50 | 16 | 8 | 4 | 37 |
| Babies | | | | | |
| M:F | 1.5 | 0.8 | 0.6 | 1 | 2.6 |
| Gestational | 28.8 ± 2.3 | 25.9 ± 1.9† | 26.4 ± 1.9† | 27.0 ± 4.3 | 26.0 ± 1.3† |
| Age (wk)* | | | | | |
| Birth | 1145 ± 259 | 875 ± 254† | 824 ± 249 | 981 ± 409† | 900 ± 197† |
| Weight | | | | | |
| (g)* | | | | | |
| Day of Life | — | 19 (8-72) | 19 (10-48) | 13 (6-20) | 21 (5-62) |
| of Blood | | | | | |
| Culture ** | | | | | |

*mean ± standard deviation
** median (range)
†P < 0.05 compared to infants with uncomplicated clinical courses as determined by post- hoc testing (Dunnet method).

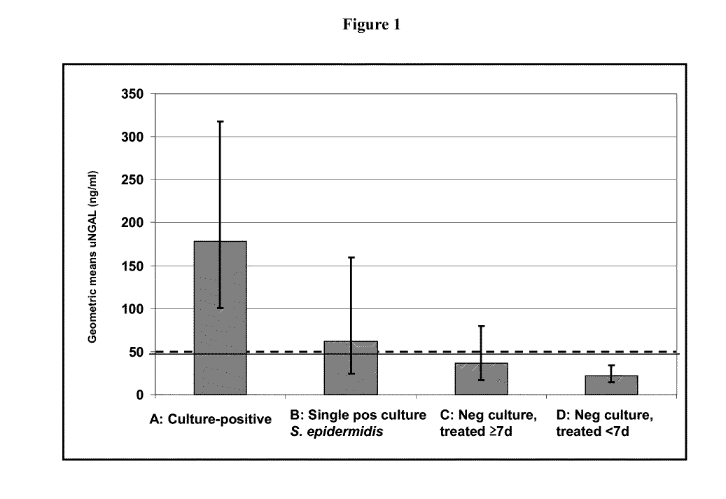

Mean and 95% CI of uNGAL concentrations (ng/ml) for the four categories of suspected late-onset sepsis on day 0 were as follows: group A (culture-positive sepsis): 178 (100, 317); group B (single positive blood culture for S. epidermidis): 62 (24, 160); group C (negative blood cultures with ≧7 days of antibiotic therapy): 37 (17, 79); and group D (negative culture with <7 days of antibiotic therapy): 22 (14, 35). These are displayed in FIG. 1, along with the upper bound of the 95% confidence limit of the reference range (55 ng/ml). Mean uNGAL concentration was significantly higher in the culture positive sepsis group (group A) than in the group with negative cultures and treatment <7 days (group D). Confidence intervals for groups A and D, respectively, lay well above and below the upper bound of the 95% confidence interval of the reference range. A linear regression model controlling for birth weight and gender demonstrated significant differences between values of mean log (uNGAL) in group A and each of the other three groups. On day −1, the mean uNGAL value of group A was lower than on day 0 [57 (31, 106) vs. 178 (100, 317), for mean and 95% CI in ng/ml, respectively], but still differed significantly from the value of group D on day −1 in the linear regression model controlling for birth weight and gender. Of note, the three infants with late-onset culture positive sepsis, who developed concurrent AKI, had uNGAL concentrations in the range 100-1500 ng/ml on day 0.

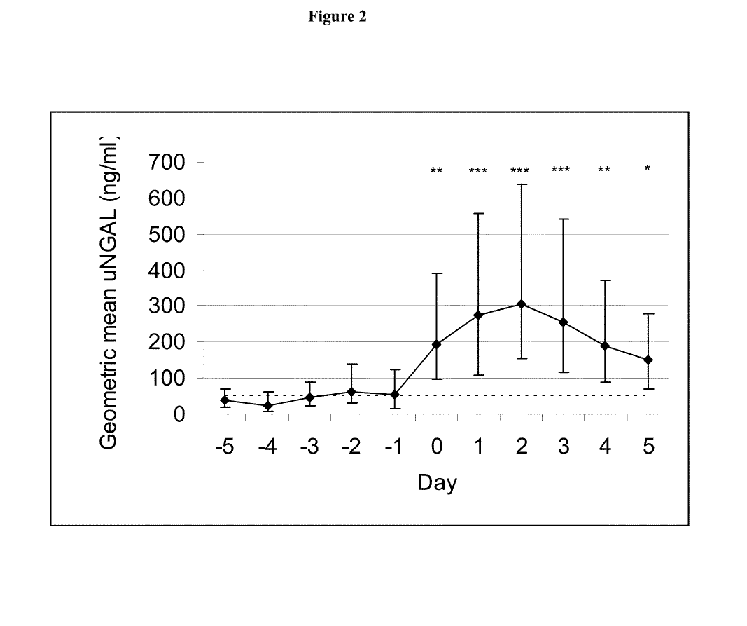

For infants in group A, the pattern of uNGAL concentration over time relative to the day when blood cultures were obtained (day 0) is shown in FIG. 2. Mean uNGAL concentrations on days −5 to −1 were similar. The uNGAL concentration on day 0 was significantly greater than on day −1, as were the concentrations on day +1 to +5 (n=16; p <0.05 to <0.005). In contrast, in group D infants, mean uNGAL on day 0 was not elevated compared to the reference range, nor to the day −1 level (n=37; p=0.60).

ROCs were constructed to evaluate the ability of uNGAL to discriminate between episodes of confirmed sepsis (group A) and episodes with negative blood culture and treatment <7 d (group D) on DOL 0, for sexes combined (shown in FIG. 3) and for males and females separately. AUC values and their 95% CIs for sexes combined, for males, and for females are 0.87 (0.77-0.97), 0.87 (0.69-1.0), and 0.83 (0.65-1.0), respectively. When uNGAL values of 75 ng/mL for males (<99th percentile of male reference range) and 130 ng/mL for females are used (˜99th percentile of female reference range) and combined into a single gender dependent indicator variable, the resulting test has the following characteristics: odds ratio, 15.5 (3.71-65); X2, 17.2 (p<0.0001); sensitivity, 12/16 =0.75 (0.51-0.89); specificity, 31/37=0.84 (0.69-0.92); positive predictive value (PPV), 12/18=0.67 (0.44-0.84); negative predictive value (NPV), 31/35=0.89 (0.74-0.95).

### Discussion

Infants with culture proven sepsis expressed uNGAL at levels approximately 30 fold higher than healthy infants. Moreover, uNGAL was elevated on the same day that blood cultures were obtained. That uNGAL concentration was not elevated in patients whose positive cultures were presumed to be due to contamination or were negative suggests that the uNGAL assay may have strong negative predictive value for sepsis. The time course of elevation of uNGAL in relation to the day the blood cultures were obtained demonstrates the rapid expression of uNGAL at the time sepsis was first suspected clinically and its persistence and decline thereafter with treatment.

In the newborn population uNGAL has several properties that complements or improves upon those of other available biomarkers of newborn sepsis. A study in a pediatric population with bacterial infection comparing the kinetics of C-reactive protein (CRP), a widely used biomarker of acute inflammation, with serum NGAL found a concomitant rise of both the markers with bacterial infection; however, NGAL was noted to be a better marker for monitoring antibiotic treatment, as its serum levels were more rapidly reversible than CRP over the course of treatment. Fjaertoft et al. Acta Pediatr, 94: 661-666 (2005). In these data, the rapid decrement of uNGAL levels following treatment is also evident (FIG. 2). The data also shows that, at least in some cases, uNGAL is expressed earlier in true sepsis than when the signs of disease are evident in the infant. Indeed, the mean uNGAL value on day −1 in group A infants lies above the upper bound of normal and significantly exceeds the mean day −1 value of infants whose cultures proved negative (group D).

uNGAL fills the needed role as a marker for late-onset sepsis. Thus, this study focused on suspected sepsis arising after 4 days of age; all the cases of proven sepsis met the definition of late-onset disease. Another advantage is that uNGAL can be detected in a very small amount of urine collected under non-invasive and non-sterile conditions and, in contrast to CRP, requires no blood specimen. This property of uNGAL is important for tiny premature infants in whom vascular access is difficult and whose limited blood volume can be easily depleted by specimen drawing.

The use of uNGAL can be critical in preventing morbidity and mortality in this fragile population. Since there is no gold standard for diagnosing sepsis the day it occurs and uNGAL can detect sepsis better than clinical judgment, uNGAL should be measured as soon as the first non-specific symptoms appear. In conclusion, uNGAL can be used for the early diagnosis of sepsis in VLBW infants.

That uNGAL concentration was not elevated in patients whose positive cultures were presumed to be due to contamination or were negative, suggests that the uNGAL assay may have a useful negative predictive value for sepsis. The time course of elevation of uNGAL in relation to the day the blood cultures were obtained demonstrates the rapid expression of uNGAL at the time sepsis was first suspected clinically and its persistence and decline thereafter with treatment.

According to clinical studies in adults and children, serum NGAL is elevated with sepsis, but our results suggest that the kidney also responds to sepsis by up-regulating uNGAL production. It is unlikely that much of this protein found in the urine derives from circulating NGAL filtered by the glomerulus, given that in animal model serum NGAL is cleared by the proximal tubule. Although it is known that tubule expression of NGAL is up-regulated by diseases that also reduce the GFR, in our study only three out of 16 infants had a concomitant episode of reduced GFR as defined by increased and sustained plasma creatinine by <0.3 mg/dL. This suggests that, when sepsis was first suspected, uNGAL was up-regulated directly by bacterial products themselves or by circulating inflammatory mediators acting on the distal tubule where NGAL is synthesized, even in the absence of an effect of these agents on glomerular function. NGAL is known to be selectively up-regulated by IL-1β and by agonists of TLR TLR4 and TLR2 in epithelial cells. Hence, the expression of uNGAL in the setting of sepsis without loss of GFR is consistent with its known molecular signal transduction. Indeed, because of the robust biologic response of NGAL to IL-1β and TLR agonists, a great increase in concentration of uNGAL is expected in infants with culture-positive sepsis.

uNGAL is best known for its association with ischemia, cytotoxicity, and sepsis. In a study of more than 650 adults presenting to an inner city Emergency Department, sepsis produced the highest levels of uNGAL especially when it was associated with a reduced GFR (Nickolas TL, O'Rourke M J, Yang J, Sise M E, Canetta P A, Barasch N, Buchen C, Khan F, Mori K, Giglio J, Devarajan P, Barasch J M 2008., Sensitivity and specificity of a single emergency department measurement of urinary neutrophil gelatinase-associated lipocalin for diagnosing acute kidney injury. Ann Intern Med 148:810-819). Indeed, the uNGAL concentration in three of our patients with culture positive sepsis associated with reduced GFR was as much as eight times higher than that of the other 13 infants with culture positive sepsis, who had no associated increase in serum creatinine. However, the fact that the rise in uNGAL generally occurred without meeting our criteria for reduced GFR, confirms the direct effect of sepsis on the renal tubule.

Zappitelli et al. (Zappitelli M, Washburn K K, Arikan A A, Loftis L, Ma Q, Devarajan P, Parikh C R, Goldstein S L 2007 Urine neutrophil gelatinase-associated lipocalin as an early marker of acute kidney injury in critically ill children: a prospective cohort study. Crit Care 11:R84) studied a heterogeneous pediatric population (age 1 mo to 21 y) and found no difference in uNGAL concentration in septic patients with or without positive blood culture. uNGAL concentration was elevated in septic patients only in presence of AKI. However, their definition of sepsis included patients diagnosed with sepsis either on admission or discharge. This is a very broad definition including children with proven sepsis and children in whom sepsis was clinically suspected or was ruled out during the admission. In contrast, the focus of our study was testing the ability of uNGAL to identify infants with episodes of culture proven sepsis.

In the newborn population uNGAL has several properties that potentially may complement or improve on those of other available biomarkers of newborn sepsis. A study in a pediatric population with bacterial infection comparing the kinetics of CRP, a widely used biomarker of acute inflammation and serum NGAL found a concomitant rise of both the markers with bacterial infection, however, NGAL was noted to be a better marker for monitoring antibiotic treatment, because its serum levels were more rapidly reversible than CRP during the course of treatment (Fjaertoft G, Foucard T, Xu S, Venge P 2005 Human neutrophil lipocalin (HNL) as a diagnostic tool in children with acute infections: a study of the kinetic. Acta Paediatr 94:661-666). In the present data, the rapid decrement of uNGAL levels after treatment is also evident (FIG. 2). The data also suggests that, at least in some cases, uNGAL is expressed earlier in true sepsis than the signs of disease are evident in the infant. Indeed, the mean uNGAL value on day 1 in group A infants lies above the upper bound of normal and significantly exceeds the mean day 1 value of infants whose cultures proved negative (group D). The time scale of our study is measured in days; however, septic premature newborns may deteriorate over hours, sometimes minutes. Thus, more frequent uNGAL specimen collection, in relation to the presentation of disease signs and the timing of the blood cultures, is contemplated by the present invention.

In the newborn CRP is sometimes used as a negative predictor of early-onset sepsis; in other words, a nonelevated CRP result bolsters the case for stopping treatment in a newborn whose blood culture is negative and whose signs and symptoms may be ambiguous. The role of CRP in identifying nosocomial sepsis (occurring after 72 h age) is not well-defined. The present data suggests that uNGAL may fill the needed role as a marker for late-onset disease. Another potential advantage is that uNGAL can be detected in a very small amount of urine collected under noninvasive and nonsterile conditions and, in contrast to CRP, requires no blood specimen. This property of uNGAL is important for tiny premature infants in whom vascular access is difficult and whose limited blood volume can be easily depleted by specimen drawing.

Based on the data presented herein, when uNGAL values of 75 ng/mL for males and 130 ng/mL for females are combined into a single indicator variable, the sensitivity, specificity, and NPV are high. It is possible to generate a different balance of sensitivity and specificity by altering our cutoff values depending on the clinical circumstances in which the test may be used. Such variations are within the scope of the invention.

### Example 2

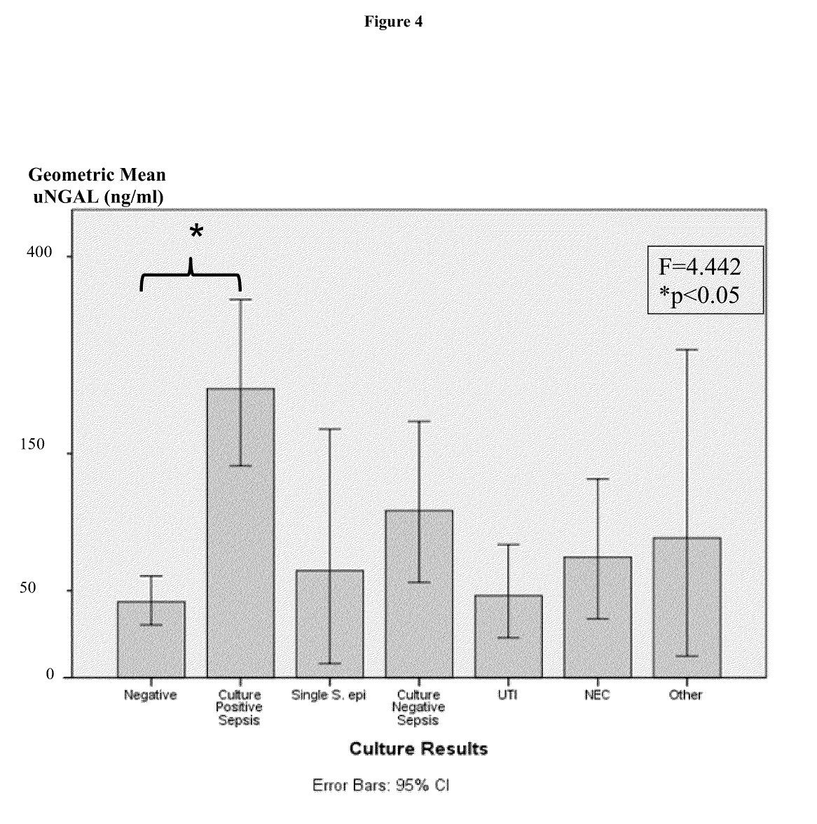

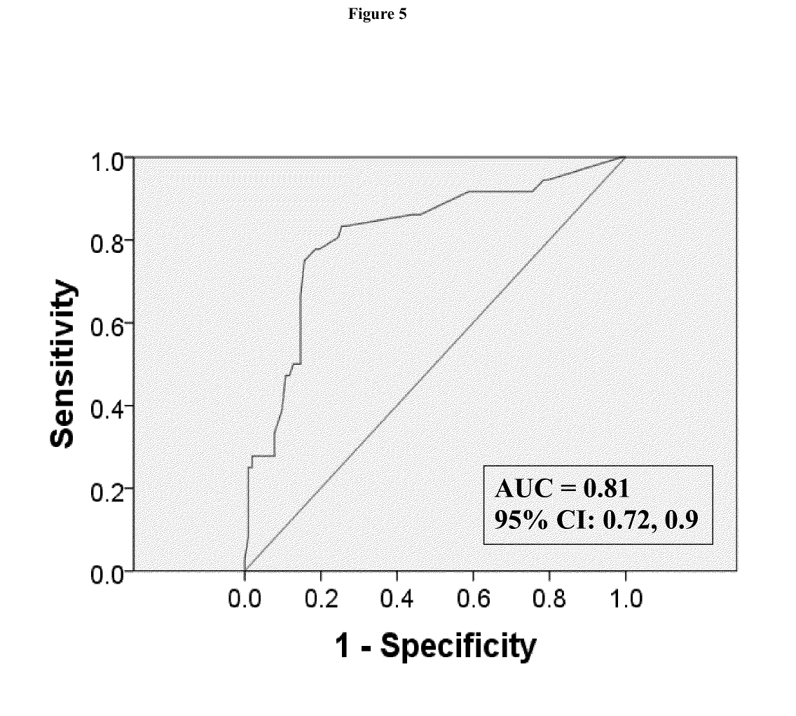

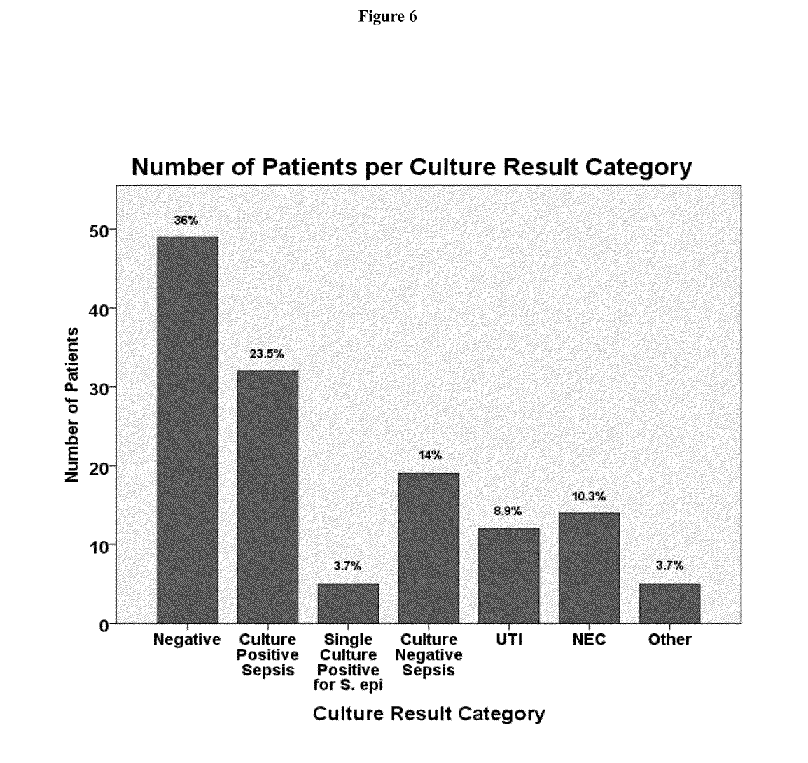

A prospective observational study was conducted to evaluate the role of urinary NGAL as a potential biomarker for late onset sepsis among infants hospitalized in the neonatal intensive care unit. Daily urine collections were obtained from preterm and full term neonates of all gestational ages and birth weights undergoing a sepsis evaluation at greater than or equal to 72 hours of age. The reference range for uNGAL in neonates was 5-120 ng/ml with a 95th percentile value of 50 ng/ml. Late onset sepsis evaluation consisted of performing/obtaining a complete blood count (CBC), one or more blood culture(s), two CRP levels, urine cultures, and cerebral-spinal fluid (CSF) culture(s)—if indicated. Pathogens detected included gram positive bacteria (51% -including Methicillin-sensitive Staphylococcus aureus/MSSA (8), Staphylococcus epidermidis (8), Group B streptococcal septicemia/GBS (1), Methicillin-resistant Staphylococcus aureus/MRSA (1), and actinomyces (1), gram negative bacteria (41%—including Klebsiella (10), Escherichia coli (4), and enterobacter (1), and fungi (8%—including Candida (3). For uNGAL measurement 500-1000 μl of urine was obtained, centrifuged within 12 hours of collection, and the supernatant stored at −80° C. The urine samples were analyzed via western blot for uNGAL values. There were 179 eligible patients with 368 eligible episodes. Of these, 136 patients (76%) with 214 episodes had uNGAL levels obtained (58.2%), while for 43 patients (24%) with 154 episodes uNGAL levels were not obtained (41.8%) for various reasons including parental refusal and oliguria/anuria. The results of the study are illustrated in FIGS. 4, 5 and 6, and Tables 2, 3, 4 and 5 below. The results demonstrated that uNGAL levels were significantly higher at the time of sepsis evaluation in patients of all gestational ages (Gas) and all birth weights (BWs) with culture proven late-onset sepsis, and that at a cutoff value of ≧50 ng/ml, uNGAL discriminates between positive and negative blood culture status with a sensitivity and negative predictive value (NPV) of 86% and 92%, respectively. This NPV makes uNGAL a very useful marker for late onset sepsis. When compared to the initial CRP value at the time of a sepsis evaluation, uNGAL demonstrated a higher sensitivity and NPV for culture positive sepsis and a lower number-needed-to-treat.

TABLE 2
A comparison of uNGAL cutoff values to CRP.

| | UNGAL | | | | | |
|---|---|---|---|---|---|---|
| | Cutoff | | | | | |
| Category | (ng/ml) | Sensitivity | Specificity | PPV | NPV | NNT |
| Culture | 20 | 94 | 22 | 30 | 92 | 5 (2, 40) |
| Positive vs | 50 | 86 | 55 | 40 | 92 | 3 (2, 5) |
| Culture | 165 | 67 | 85 | 62 | 88 | 2 (2, 3) |
| Negative | | | | | | |
| CRP #1 | — | 83 | 61 | 32 | 94 | 2 |
| (>1 mg/dl) | | | | | | |
| *CRP #1 | — | 62 | 69 | 44 | 82 | 4 (3, 8) |
| (>1 mg/dl) | | | | | | |

TABLE 3
A comparison of uNGAL cutoff values to CRP.

| | UNGAL | | | | | |
|---|---|---|---|---|---|---|
| | Cutoff | | | | | |
| Category | (ng/ml) | Sensitivity | Specificity | PPV | NPV | NNT |
| Culture | 20 | 93 | 21 | 38 | 85 | 4 (2, 24) |
| Positive | 50 | 83 | 56 | 49 | 86 | 3 (2, 5) |
| & Clinical | 165 | 57 | 85 | 66 | 79 | 2 (2, 3) |
| Sepsis vs | | | | | | |
| Culture | | | | | | |
| Negative | | | | | | |
| CRP #1 | — | 82 | 67 | 51 | 90 | 3 |
| (>1 mg/dl) | | | | | | |

TABLE 4
ANOVA of log uNGAL Values.

| | Sum of | | Mean | | |
|---|---|---|---|---|---|
| | Squares | Df | Square | F | Sig. |
| Between | 43.038 | 6 | 7.173 | 4.442 | .000 |
| groups | | | | | |
| Within | 206.713 | 128 | 1.615 | | |
| groups | | | | | |
| Total | 249.751 | 134 | | | |

TABLE 5
Post-Hoc Analysis Using Dunnett t-test.

| | | Mean | | | 95% CI - | 95% CI - |
|---|---|---|---|---|---|---|
| | (J) | Difference | Std. | | lower | upper |
| (I) Results | Results | (I − J) | Error | Sig. | bound | bound |
| Cx | 0 | 1.40339* | .291164 | .000 | .6277 | 2.1791 |
| Positive | | | | | | |
| Sepsis | | | | | | |
| Single | 0 | .17149 | .59661 | 1.000 | −1.4153 | 1.7583 |
| Staph epi | | | | | | |
| Cx | 0 | .61190 | .35025 | .384 | −.3197 | 1.5435 |
| Negative | | | | | | |
| Sepsis | | | | | | |
| UTI | 0 | −.02101 | .39646 | 1.000 | −1.0755 | 1.0335 |
| NEC | 0 | .21014 | .38511 | .994 | −.8141 | 1.2344 |
| Other | 0 | .16173 | .59661 | 1.000 | −1.4251 | 1.7485 |

*The mean difference is significant at the 0.05 level. (Note - Dunnett t-tests treat one group as a control and compare all other groups against it).

### REFERENCES

1. Stoll B J, Hansen N, Fanaroff A A, Wright L L, Carlo W A, Ehrenkranz R A, et al. Late-onset sepsis in very low birth weight neonates: the experience of the NICHD Neonatal Research Network. Pediatrics 2002; 110: 285-91.

2. Bizzarro M J, Raskind C, Baltimore R S, Gallagher P G. Seventy-five years of neonatal sepsis at Yale: 1928-2003. Pediatrics 2005; 116(3): 595-602.

3. Graham P L 3rd, Begg M D, Larson E, Della-Latta P, Allen A, Saiman L. Risk factors for late onset gram-negative sepsis in low birth weight infants hospitalized in the neonatal intensive care unit. Pediatr Infect Dis J 2006; 25(2): 113-7.

4. Fanaroff A A, Stoll B J, Wright L L, et al. NICHD Neonatal Research Network. Trends in neonatal morbidity and mortality for very low birthweight infants. Am J Obstet Gynecol 2007; 196(2): 147.e1-8.

5. Brodie SB, Sands KE, Gray JE, et al. Occurrence of nosocomial bloodstream infections in six neonatal intensive care units. Pediatr Infect Dis J2000; 19: 56-65.

6. Apisarnthanarak A, Holzmann-Pazgal G, Hamvas A, Olsen M A, Fraser V J. Antimicrobial use and the influence of inadequate empiric antimicrobial therapy on the outcomes of nosocomial bloodstream infections in a neonatal intensive care unit. Infect Control Hosp Epidemiol 2004; 25(9): 735-41.

7. Rubin L G, Sánchez P J, Siegel J, Levine G, Saiman L, Jarvis W R, the Pediatric Prevention Network. Evaluation and treatment of neonates with suspected late-onset sepsis: a survey of neonatologists' practices. Pediatrics 2002; 110(4): e42.

8. Calil R, Marba S T, von Nowakonski A, Tresoldi A T. Reduction in colonization and nosocomial infection by multiresistant bacteria in a neonatal unit after institution of educational measures and restriction in the use of cephalosporins. Am J Infect Control 2001; 29(3): 133-8.

9. Benitz W E, Han M Y, Madan A, Ramachandra P. Serial serum C-reactive protein levels in the diagnosis of neonatal infection. Pediatrics 1998; 102(4): E41.

10. Hengst J M. The role of C-reactive protein in the evaluation and management of infants with suspected sepsis. Adv Neonatal Care 2003; 3(1): 3-13.

11. N g PC, Lam H S. Diagnostic markers for neonatal sepsis. Curr Opin Pediatr 2006; 18(2): 125-31.

12. Mishra J, Ma Q, Prada A, et al. Identification of neutrophil gelatinase-associated lipocalin as a novel early urinary biomarker for ischemic renal injury. J Am Soc Nephrol 2003;14(10): 2534-43.

13. Schmidt-Ott K M, Mori K, Kalandadze A, et al. Neutrophil gelatinase-associated lipocalin-mediated iron traffic in kidney epithelia. Curr Opin Nephrol Hypertens 2006; 15: 442-449.

14. Schmidt-Ott K M, Mori K, Li J Y, et al. Dual action of neutrophil gelatinase-associated lipocalin. J Am Soc Nephrol 2007; 18: 407-413.

15. Mori K, Lee H T, Rapoport D, et al. Endocytic delivery of lipocalin-siderophore-iron complex rescues the kidney from ischemia-reperfusion injury. J Clin Invest 2005; 115: 610-621.

16. Fjaertoft G, Foucard T, Xu S, Venge P. Human neutrophil lipocalin (HNL) as a diagnostic tool in children with acute infections: a study of the kinetic. Acta Pediatr 2005; 94: 661-666.

17. Flo TH, Smith K D, Sato S, et al. Lipocalin 2 mediates an innate immune response to bacterial infection by sequestrating iron. Nature 2004; 432: 917-21.

18. Parravicini E, Lorenz J M, Nemerofsky S L, O'Rourke M, Barasch J, Bateman D. Reference range of urinary neutrophil gelatinase-associated lipocalin in very low birth weight infants: preliminary data. Am J Perinatol 2009; 26(6): 437-40.

19. Huynh T K, Bateman D A, Parravicini E, et al. Reference Values of Urinary Neutrophil Gelatinase-Associated Lipocalin in Very Low Birth Weight Infants. Pediatr Res 2009 Aug. 12 [Epub ahead of print].

20. Mishra J, Dent C, Tarabishi R, et al. Neutrophil gelatinase-associated lipocalin (NGAL) as a biomarker for acute renal injury after cardiac surgery. Lancet 2005; 365: 1231-8.

21. Cowland J B, Sorensen O E, Sehested M, Borregaard N. Neutrophil gelatinase-associated lipocalin is up-regulated in human epithelial cells by IL-1 beta, but not by TNF-alpha. J Immunol 2003; 171(12): 6630-9.

22. Nickolas TL, O'Rourke M J, Yang J, et al Sensitivity and specificity of a single emergency department measurement of urinary neutrophil gelatinase-associated lipocalin for diagnosing acute kidney injury. Ann Intern Med 2008; 148(11): 810-9.

23. Akcan-Arikan A, Zappitelli M, Loftis L L, Washburn K K, Jefferson L S and Goldstein S L. Modified RIFLE criteria in critically ill children with acute kidney injury. Kidney Int 2007; 71: 1028-1035.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention.

What is claimed is:

1. A method for determining whether a very low birth weight (VLBW) infant has sepsis, the method comprising determining the concentration of NGAL protein in a urine sample from a very low birth weight infant, wherein a concentration of NGAL in the urine sample that exceeds a threshold amount indicates that the infant has sepsis, and wherein a concentration of NGAL in the urine sample that is less than the threshold amount indicates that the infant does not have sepsis.

2. The method of claim 1, wherein the threshold amount is between about 60 ng/ml and about 150 ng/ml.

3. The method of claim 1, wherein the threshold amount is about 75 ng/ml in males and about 130 ng/ml in females.

4. The method of claim 1 comprising contacting the urine sample with an agent that binds to an NGAL protein.

5. The method of claim 4, wherein the agent is an NGAL antibody.

6. The method of claim 1, further comprising subsequently treating the VLBW infant with antibiotics.

7. A method for determining whether a VLBW infant has late onset sepsis, the method comprising determining the concentration of NGAL protein in a urine sample from a very low birth weight infant, wherein a concentration of NGAL in the urine sample that exceeds a threshold amount indicates that the infant has late onset sepsis, and wherein a concentration of NGAL in the urine sample that is less than the threshold amount indicates that the infant does not have late onset sepsis.

8. The method of claim 7, wherein the threshold amount is between about 60 ng/ml and about 150 ng/ml.

9. The method of claim 7, wherein the threshold amount is about 75 ng/ml in males and about 130 ng/ml in females.

10. The method of claim 7, comprising contacting the urine sample with an agent that binds to an NGAL protein.

11. The method of claim 10, wherein the agent is an NGAL antibody.

12. The method of claim 7, further comprising subsequently treating the VLBW infant with antibiotics.

13. A method for distinguishing between true-positive sepsis and false-positive sepsis in a VLBW infant, the method comprising determining the concentration of NGAL protein in a urine sample from a VLBW infant with sepsis, wherein a concentration of NGAL in the urine sample that exceeds a threshold amount indicates that the infant has true-positive sepsis, and wherein a concentration of NGAL in the urine sample that is less than the threshold amount indicates that the infant has true-positive sepsis.

14. The method of claim 13, wherein the threshold amount is between about 60 ng/ml and about 150 ng/ml.

15. The method of claim 13, wherein the threshold amount is about 75 ng/ml in males and about 130 ng/ml in females.

16. The method of claim 13 comprising contacting the urine sample with an agent that binds to an NGAL protein.

17. The method of claim 16, wherein the agent is an NGAL antibody.

18. The method of claim 13, further comprising subsequently treating the VLBW infant with antibiotics.

19. The method of claim 13, wherein if the NGAL level in the urine sample exceeds the threshold amount, the VLBW infant is subsequently treated with an antibiotic regimen suitable for treatment of late onset sepsis.

20. The method of claim 13, wherein if the NGAL level in the urine sample is less than the threshold amount, the VLBW infant is not treated with antibiotics, or antibiotic therapy is halted.

21. A method for monitoring the progression of sepsis in a VLBW infant, the method comprising:
(a) obtaining a first urine sample from a VLBW infant at a first time point;
(b) obtaining a second urine sample from the VLBW infant at a second time point that is after the first time point; and
(c) determining the amount of NGAL protein in the first and second urine samples, wherein an amount of NGAL protein in the first urine sample that exceeds the amount in the second urine sample indicates that the sepsis is improving, and
wherein an amount of NGAL protein in the second urine sample that exceeds the amount in the first urine sample indicates that the sepsis is worsening.

22. The method of claim 21, comprising contacting the urine sample with an agent that binds to an NGAL protein.

23. The method of claim 22, wherein the agent is an NGAL antibody.

24. The method of claim 21, further comprising subsequently treating the VLBW infant with antibiotics.

25. The method of claim 21, wherein the first urine sample is obtained before initiation antibiotic treatment, and the second urine sample is obtained after initiation of antibiotic treatment.

26. The method of claim 21, wherein both the and second urine samples are obtained after initiation of antibiotic treatment.

27. The method of claim 25 or 26, further comprising subsequently adjusting the infant's treatment regimen.

28. A diagnostic kit for determining whether a VLBW infant has sepsis, the kit comprising: (a) a device for detecting NGAL protein in the urine; (b) a positive control containing NGAL protein; and (c) instructions indicating a threshold level of NGAL above which a diagnosis of sepsis can be made in a VLBW infant.

29. The diagnostic kit of claim 28, wherein the device for detecting NGAL protein in the urine comprises an antibody that binds to the NGAL protein.

30. The diagnostic kit of claim 28, wherein the device for detecting NGAL protein in the urine is an ELISA plate, a urine dipstick, or a test strip.
